(19) United States
(12) Patent Application Publication
(10) Pub. No.: US 2016/0051513 A1
(43) Pub. Date: Feb. 25, 2016

(54) USE OF 4-BENZAMIDE IN THE TREATMENT OF NEUROPATHIC PAIN

(71) Applicant: LES LABORATOIRES SERVIER, Suresnes (FR)

(72) Inventors: Aurore SORS, Paris (FR); Annette MERDES, Munchen (DE)

(21) Appl. No.: 14/778,628

(22) PCT Filed: Mar. 21, 2014
(86) PCT No.: PCT/FR2014/050659

§ 371 Date: Sep. 21, 2015

(30) Foreign Application Priority Data
Mar. 22, 2013 (FR) 1352569

(51) Int. Cl. A61K 31/403; A61K 31/195; A61K 31/167; A61K 31/197

(52) U.S. Cl. CPC A61K 31/403; A61K 31/197; A61K 31/195; A61K 31/167

(57) ABSTRACT

Use of 4-{3-[cis-hexahydrocyclopenta[c]pyrrol-2(1H)-yl]propoxy}benzamide of formula (I):

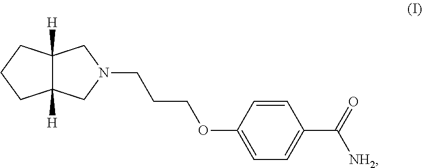 [SMILES: NC(=O)c1ccc(OCCCN2C[C@H]3CCC[C@H]3C2)cc1]

or an addition salt thereof with a pharmaceutically acceptable acid or base, in the treatment of neuropathic pain and of cognitive disorders, depression and anxiety associated with that pain.

The present invention relates to 4-{3-[cis-hexahydrocyclopenta[c]pyrrol-2(1H)-yl]-propoxy}benzamide of formula (I):

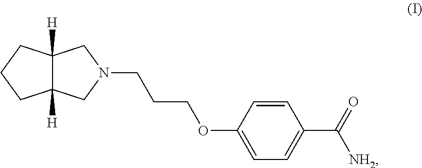 [SMILES: NC(=O)c1ccc(OCCCN2C[C@H]3CCC[C@H]3C2)cc1]

or an addition salt thereof with a pharmaceutically acceptable acid or base, for use in the treatment of neuropathic pain and of cognitive disorders, depression and anxiety associated with that pain.

4-{3-[cis-Hexahydrocyclopenta[c]pyrrol-2(1H)-yl]propoxy}benzamide has the characteristic of interacting with central histaminergic systems in vivo. These properties provide it with activity in the central nervous system and, more especially, in the treatment of cognitive deficiencies associated with cerebral aging and with neurodegenerative diseases.

4-{3-[cis-Hexahydrocyclopenta[c]pyrrol-2(1H)-yl]propoxy}benzamide, its preparation and its therapeutic use have been described in Patent Application WO2005/089747.

More specifically, the present Application relates to antalgic properties of 4-{3-[cis-hexahydrocyclopenta[c]pyrrol-2(1H)-yl]propoxy}benzamide, or of addition salts thereof with a pharmaceutically acceptable acid or base, and their potential use in the treatment of neuropathic pain.

Neuropathic pain is chronic pain caused by a lesion or disorder affecting the somatosensory system (Treede et al. Neurology, 2008, 70 : 1630-1635). A distinction is made between two types: (i) peripheral neuropathic pain, which is the more frequent and due to lesions located in the peripheral nervous system (roots, plexuses, trunks, fibres); and (ii) central neuropathic pain resulting from lesions located in the central nervous system (brain, spinal cord). The prevalence of neuropathic pain is estimated to be 7-8% of the general population in Europe.

Among peripheral neuropathic pain there may be mentioned pain of metabolic origin (diabetes, hypothyroidism . . . ), post-traumatic origin, post-surgical origin, toxic origin (associated with alcohol consumption or with the administration of anticancer or antiretroviral medicaments) or infectious origin (associated with human immunodeficiency virus HIV, or with varicella zoster virus VZV), pain associated with hereditary neuropathies (Fabry disease, amyloidosis) and with trigeminal neuralgia, and also pain associated with inflammatory diseases (Guillain-Barr syndrome), with certain cancers, and with autoimmune or haematological disorders (Sjogren's syndrome). Central neuropathic pain occurs most frequently as a result of lesions having a vascular (post-CVA), tumour, trauma or inflammatory origin. Such pain may also be the consequence of neurodegenerative disorders.

The clinical symptoms observed in patients suffering from neuropathic pain may be likened to spontaneous sensations of burning, cold, vice-like pain, compression, electric shocks, shooting pains or stabbing pains. Other sensations are caused by mechanical stimuli (allodynia/hyperalgesia caused by friction, light touching or vibration) or thermal stimuli (allodynia/hyperalgesia caused by heat or cold). Furthermore, abnormal sensations such as tingling or pins and needles are also reported by patients (paresthesia/dysesthesia).

The comorbidities most frequently associated with neuropathic pain are depression, anxiety, cognitive disorders and also sleep disorders which have on impact on quality of life (loss of employment, reduction in day-to-day activities, reduced enjoyment of life). Several clinical studies have shown that patients suffering from chronic pain are more susceptible to developing anxious and depressive symptoms, often associated with cognitive deficits. Work done in models of neuropathic pain in rats has also shown that the pain symptoms are associated with pyschobehavioural and cognitive disorders (Roeska et al. Pain, 2008, 139(2):349-57; Roeska et al. Neurosci Lett., 2009, 464(3):160-4; Grégoire et al. Pain, 2012, 153(8):1657-63). Interestingly, Alba-Delgado et al. Biol. Psychiatry, 2013, 73(1):54-62 have shown that the neuropathic pain brought about long-term in the rat after constriction of the sciatic nerve has the consequence of the animals' inability to face up to stressful situations, causing depressive and anxious behaviours.

Currently, the treatments customarily recommended for dealing with neuropathic pain are anticonvulsants such as pregabalin or gabapentin, antidepressants such as duloxetine or amitriptyline, opioid agonists (morphine, tramadol) or local anaesthetics (lidocaine, capsaicin). Unfortunately, many patients (on average 50%) suffering from neuropathic pain do not respond satisfactorily to the existing treatments. Furthermore, their effect is sometimes not felt until after several weeks. These treatments moreover cause troublesome secondary effects, the most commonly recorded being drowsiness, sedation, dizziness, ataxia, appetite disorders, orthostatic hypotension and nausea. They may also be responsible for a large number of drug interactions. Finally, the mode of administration of the current treatments, very often necessitating repeated daily doses, can present problems of compliance.

In view of these factors, the search for new treatments that are more effective, better tolerated and with a mode of administration more suitable for relieving neuropathic pain is still current.

The results of the present invention show that 4-{3-[cis-hexahydrocyclopenta[c]pyrrol-2(1H)-yl]propoxy}benzamide has remarkable efficacy in various models of neuropathic pain in the rat. In addition, the effects of 4-{3-[cis-hexahydrocyclopenta[c]pyrrol-2(1H)-yl]propoxy}benzamide have been studied on the anxiety and depression associated with the neuropathic pain caused by constriction of the sciatic nerve. Finally, 4-{3-[cis-hexahydrocyclopenta[c]pyrrol-2(1H)-yl]propoxy}benzamide has a good acceptability profile in healthy volunteers and in patients suffering from Alzheimer's disease.

The invention accordingly relates to 4-{3-[cis-hexahydrocyclopenta[c]pyrrol-2(1H)-yl]-propoxy}benzamide, or an addition salt thereof with a pharmaceutically acceptable acid or base, for use in the treatment of central or peripheral neuropathic pain and of cognitive disorders, depression and anxiety associated with that pain.

More especially, the peripheral neuropathic pain concerned is metabolic, post-traumatic, post-surgical, toxic or infectious neuropathic pain, pain associated with hereditary neuropathies and with trigeminal neuralgia, and neuropathic pain associated with inflammatory diseases, with certain cancers, and with autoimmune or haematological disorders. Even more especially, diabetic neuropathic pain and toxic neuropathic pain which is associated with the administration of anticancer medicaments such as oxaliplatin are targeted.

In another embodiment of the invention, 4-{3[cis-hexahydrocyclopenta[c]pyrrol-2(1H)-yl]propoxy}benzamide, or an addition salt thereof with a pharmaceutically acceptable acid or base, may be used in the treatment of central neuropathic pain which originates from lesions of vascular, tumour, trauma or inflammatory origin or which are the consequence of neurodegenerative disorders.

Within the context of the invention, the 4-{3-[cis-hexahydrocyclopenta[c]pyrrol-2(1H)-yl]-propoxy}benzamide is preferably used in the form of an oxalate or hydrochloride.

The invention relates especially to use of 4-{3-[cis-hexahydrocyclopenta[c]pyrrol-2(1H)-yl]propoxy}benzamide hydrochloride obtained in crystalline form I as described in Patent Application EP 2532651 in the treatment of neuropathic pain and of cognitive disorders, depression and anxiety associated with that pain. Furthermore, crystalline form I of the free base, described in that same Patent Application EP 2532651, may advantageously be used in the treatment of neuropathic pain and of cognitive disorders, depression and anxiety associated with that pain.

The invention relates also to pharmaceutical compositions comprising 4-{3-[cis-hexahydrocyclopenta[c]pyrrol-2(1H)-yl]propoxy}benzamide, or an addition salt thereof with a pharmaceutically acceptable acid or base, in combination with one or more pharmaceutically acceptable excipients, for use in the treatment of neuropathic pain and of cognitive disorders, depression and anxiety associated with that pain.

An association of 4-{3-[cis-hexahydrocyclopenta[c]pyrrol-2(1H)-yl]propoxy}benzamide, or of an addition salt thereof with a pharmaceutically acceptable acid or base, with pregabalin or gabapentin or with an antidepressant also forms an integral part of the invention. Duloxetine, amitriptyline and agomelatine are among the preferred antidepressants. Associations of such a type may be used in the treatment of neuropathic pain and of cognitive disorders, depression and anxiety associated with that pain. Indeed, the co-administration of the compounds mentioned hereinabove could make it possible to better deal with the neuropathic pain of patients compared to the administration, on its own, of pregabalin, of gabapentin or of an antidepressant without, however, increasing the adverse effects associated with the treatment. The pharmaceutical compositions comprising one of the above associations also form an integral part of the invention.

In the pharmaceutical compositions according to the invention, the proportion of active ingredients by weight (weight of active ingredients over the total weight of the composition) is from 5 to 60%.

Among the pharmaceutical compositions according to the invention there will more especially be selected those that are suitable for administration by the oral, parenteral and especially intravenous, per- or trans-cutaneous, nasal, rectal, perlingual, ocular or respiratory route, more specifically tablets or drages, sublingual tablets, hard gelatin capsules, glossettes, capsules, lozenges, injectable preparations, aerosols, eye or nose drops, suppositories, creams, ointments, dermal gels etc.

Besides the active ingredient(s), the pharmaceutical compositions according to the invention comprise one or more excipients or carriers selected from diluents, lubricants, binders, disintegration agents, stabilisers, preservatives, absorbents, colorants, sweeteners, flavourings etc.

By way of non-limiting example there may be mentioned:
as diluents: lactose, dextrose, sucrose, mannitol, sorbitol, cellulose, glycerol;
as lubricants: silica, talc, stearic acid and its magnesium and calcium salts, polyethylene glycol;
as binders: magnesium aluminium silicate, starch, gelatin, tragacanth, methylcellulose, sodium carboxymethylcellulose and polyvinylpyrrolidone;
as disintegrants: agar, alginic acid and its sodium salt, effervescent mixtures.

The useful dosage regimen can be adapted according to the nature and severity of the disorder, the administration route and the age and weight of the patient. The dosage regimen ranges from 0.5 mg to 100 mg per day, in one or more administrations. Preferably, the 4-{3-[cis-hexahydrocyclopenta[c]pyrrol-2(1H)-yl]propoxy}benzamide is administered at daily doses (expressed in terms of the free base) of 2 mg, 5 mg, 15 mg, 20 mg, 30 mg and 50 mg.

### EXAMPLE A

### Effect of 4-{3-[cis-hexahydrocyclopenta[c]pyrrol-2(1H)-yl]propoxy}-benzamide on Mechanical Hyperalgesia in a Model of Diabetic Neuropathy Caused by Injection of Streptozocin

The effects of 4-{3-[cis-hexahydrocyclopenta[c]pyrrol-2(1H)-yl]propoxy}benzamide and of two reference treatments for neuropathic pain (gabapentin and pregabalin) were studied in the rat, in the model of diabetic neuropathy caused by streptozocin (STZ).

Streptozocin is a synthetic antitumour antibiotic chemically related to other nitrosoureas used in anticancer chemotherapy. It differs from the latter by the presence of a glucopyranosyl group which gives it particular affinity for the pancreas. In humans, streptozocin is used in the treatment of tumours of the pancreas.

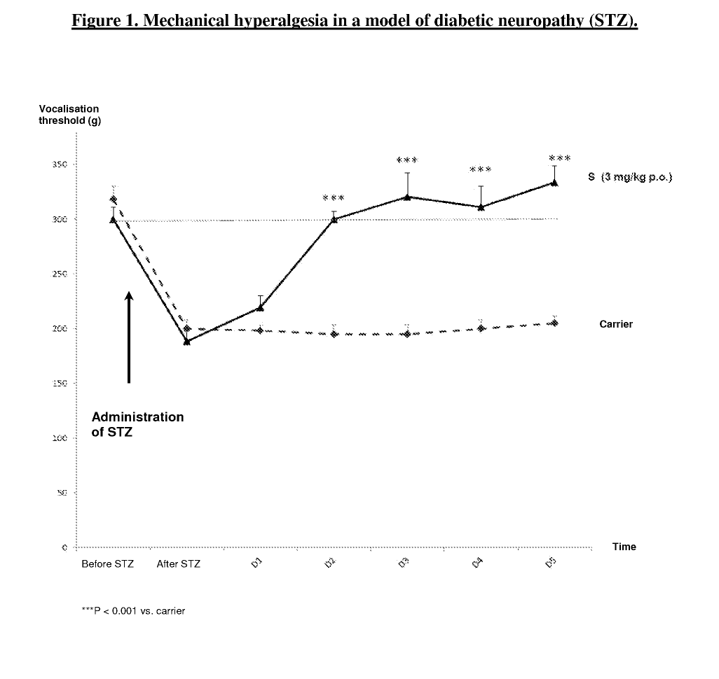

In rats, administration of high-dose STZ (>35 mg/kg) causes diabetes which is similar to hyperglycaemic diabetes mellitus in humans. This is brought about by histopathological changes in the beta cells of islets of Langerhans and is associated with neuropathic pain. This pain is made manifest especially by mechanical hyperalgesia which can be attested by the lowering of the animal's vocalisation threshold when an increasing pressure is applied to the paw (Courteix et al., Pain, 1993, 53(1):81-8). During the test, a weight is placed on the rat's paw; its weight (in grams) increases until the animal vocalises. In this experiment, 21 days after the intraperitoneal injection of STZ (72 mg/kg), the rats develop diabetes characterised especially by hyperglycaemia (controlled during the experiment by injection of insulin) and associated with mechanical hyperalgesia. Indeed, the vocalisation threshold of the rats is lowered by half compared to the threshold which those animals had prior to injection of STZ (FIG. 1). The administration of 4-{3-[cis-hexahydrocyclopenta-[c]pyrrol-2(1H)-yl]propoxy}benzamide hydrochloride at a dose of 3 mg of base per kg per os (the compound being referred to as S in FIG. 1) brings about a substantial and significant increase in the vocalisation threshold compared to animals having been given the carrier, the value of the threshold becoming the same as that of non-diabetic animals from the second day of administration. The beneficial effect of 4-{3-[cis-hexahydrocyclopenta[c]pyrrol-2(1H)-yl]propoxy}benzamide is maintained over time, when the compound is administered every day for 5 days, and is entirely comparable to the effect obtained after administration of the reference treatments, gabapentin and pregabalin.

### EXAMPLE B

### Effect of 4-{3-[cis-hexahydrocyclopenta[c]pyrrol-2(1H)-yl]propoxy}-benzamide on Mechanical Hyperalgesia in a Model of Neuropathy Caused by Constriction of the Sciatic Nerve

The effects of 4-{3-[cis-hexahydrocyclopenta[c]pyrrol-2(1H)-yl]propoxy}benzamide and of gabapentin were studied on the mechanical hyperalgesia caused in the rat by chronic constriction (CCI) of the sciatic nerve.

This model of post-traumatic neuropathic pain closely reproduces the symptoms of neuropathic pain which can be observed in humans, namely mechanical and thermal hyperalgesia, mechanical allodynia and spontaneous pain (Bennett and Xie, Pain, 1988, 33(1):87-107; Munger et al., Exp Neurol, 1992, 118(2):204-14; Kim et al., Brain Res, 1997, 113(2):200-6.).

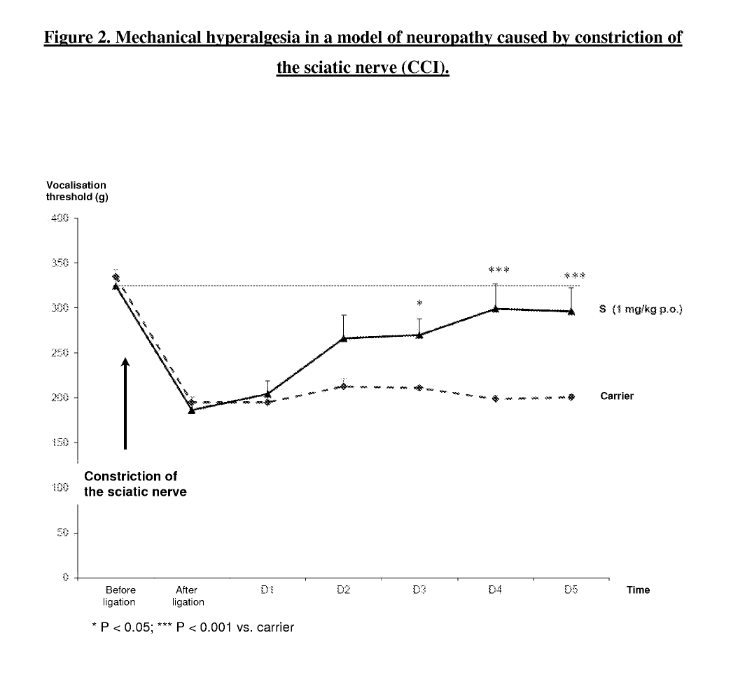

The results of this study show that, 12 days after constriction of the sciatic nerve, when an increasing pressure is applied to the paw on which the procedure has been performed, the vocalisation threshold of the rats is considerably lowered compared to the threshold they exhibited prior to the procedure. This demonstrates that marked mechanical hyperalgesia has been brought about in those animals (FIG. 2). Whereas this hyperalgesia is maintained over time in the case of those animals given the carrier, a significant anti-hyperalgesic effect is observed in those animals given 4-{3-[cis-hexahydrocyclopenta-[c]pyrrol-2(1H)-yl]propoxy}benzamide hydrochloride at a dose of 1 mg of base/kg per os (the compound being referred to as S in FIG. 2). Indeed, after just 4 days of chronic treatment, the vocalisation threshold is equivalent to that measured before the procedure, demonstrating complete reversal of the hyperalgesia. This reversal is also observed in animals given the reference treatment gabapentin.

### EXAMPLE C

### Effect of 4-{3-[cis-hexahydrocyclopenta[c]pyrrol-2(1H)-yl]propoxy}-benzamide on Thermal Allodynia in a Model of Neuropathy Caused by Oxaliplatin

The effects of 4-{3-[cis-hexahydrocyclopenta[c]pyrrol-2(1H)-yl]propoxy}benzamide and of two reference treatments for neuropathic pain (gabapentin and pregabalin) were studied on the thermal allodynia which occurs in rats after administration of oxaliplatin (OXA). Oxaliplatin is a synthetic anticancer medication from the group of agents derived from platinum, especially used in the treatment of cancers of the colon and rectum.

In clinical use, infusion of oxaliplatin very frequently causes signs of sensory neurotoxicity triggered or aggravated by exposure to cold. Indeed, 85 to 95% of patients treated with oxaliplatin develop painful symptoms, without motor dysfunction, reaching a peak 24 to 48 hours after the start of the infusion. This acute neurotoxicity is characterised by the rapid appearance of dysesthesias and paresthesias in the distal extremities, the border of the mouth and the throat. These symptoms usually disappear after 1 week.

In the rat, acute administration of oxaliplatin specifically causes cold hyperalgesia and allodynia in the subsequent 24 hours, which lasts for several days. This animal model therefore closely mimics the painful symptoms observed in humans after infusion of oxaliplatin, especially the rapid appearance of intense sensory disturbances caused by cold, of the hyperalgesic and allodynic type (Ling et al. Toxicology. 2007; 234(3):176-84).

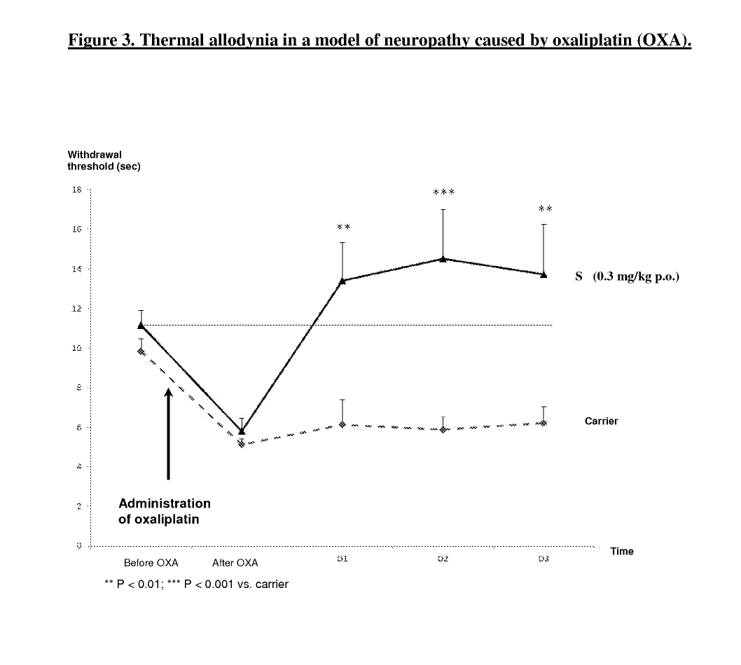

The results of this study confirm that, 36 hours after acute intraperitoneal administration of oxaliplatin (6 mg/kg), the rats develop cold allodynia, reflected by very rapid withdrawal of their tail when it is placed in a cold bath at 10° C. Indeed, this withdrawal time is, on average, half as long as that of the rats before administration of oxaliplatin (FIG. 3). Whereas this cold allodynia is maintained over time in the case of those animals given the carrier, a significant anti-allodynic effect is observed in those animals given 4-{3-[cis-hexahydrocyclopenta[c]pyrrol-2(1H)-yl]propoxy}benzamide hydrochloride at a dose of 0.3 mg of base/kg per os (the compound being referred to as S in FIG. 3), this being the case from D1, 1 hour after a single administration. The tail withdrawal time is equivalent to or greater than that measured before the procedure, demonstrating complete reversal of the allodynia. This rapid reversal is also observed in animals given the reference treatments gabapentin and pregabalin.

In conclusion, the test results show that 4-{3-[cis-hexahydrocyclopenta[c]pyrrol-2(1H)-yl]-propoxy}benzamide has notable antalgic properties in several models of neuropathic pain in the rat. Administration of the compound allowed the test animals to return to a sensitivity to pain comparable to that which they had before application of the lesion or the treatment causing the neuropathy. These results are encouraging in the clinical context in view of the good clinical acceptability profile for 4-{3-[cis-hexahydrocyclopenta[c]pyrrol-2(1H)-yl]propoxy}benzamide.

### EXAMPLE D

### Effect of 4-{3-[cis-hexahydrocyclopenta[c]pyrrol-2(1H)-yl]propoxy}-benzamide and Agomelatine, Administered on Their Own or in Association, on Mechanical Hyperalgesia in a Model of Neuropathy Caused by Constriction of the Sciatic Nerve

The effects of 4-{3-[cis-hexahydrocyclopenta[c]pyrrol-2(1H)-yl]propoxy}benzamide and agomelatine, administered on their own or in association, were studied on the mechanical hyperalgesia caused in the rat by chronic constriction (CCI) of the sciatic nerve (see Example B).

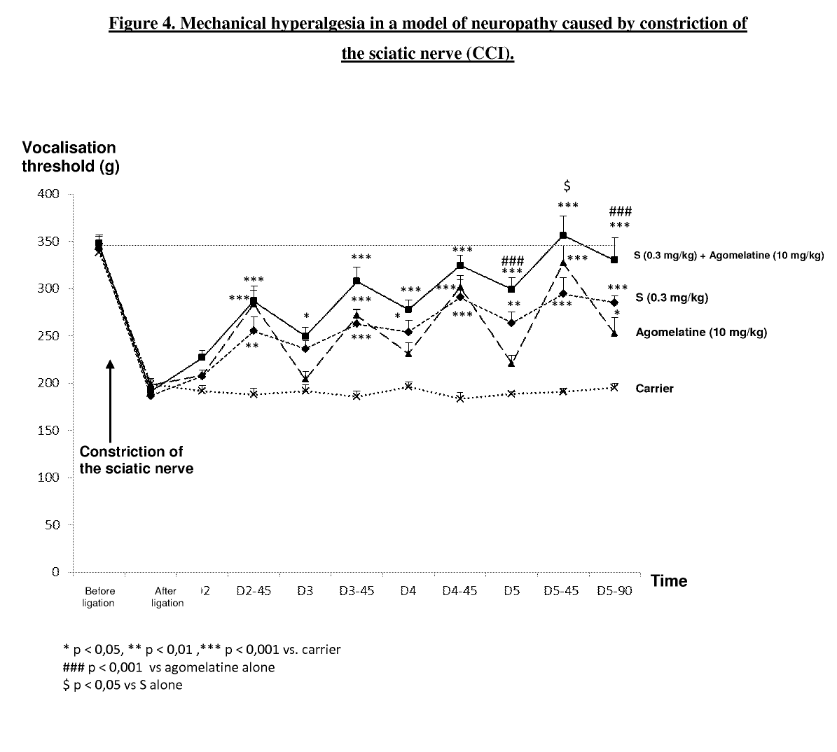

The results of this study show that, 12 days after constriction of the sciatic nerve, when an increasing pressure is applied to the paw on which the procedure has been performed, the vocalisation threshold of the rats is considerably lowered compared to the threshold they exhibited prior to the procedure (FIG. 4). This demonstrates that marked mechanical hyperalgesia has been brought about in those animals. This hyperalgesia is maintained over time in the case of those animals given the carrier.

The vocalisation threshold was measured in the animals every day for 5 days, before and 45 minutes after the administration of 4-{3-[cis-hexahydrocyclopenta[c]pyrrol-2(1H)-yl]-propoxy}benzamide hydrochloride at a dose of 0.3 mg of base/kg per os (the compound being referred to as S in FIG. 4) and agomelatine at a dose of 10 mg/kg i.p., on their own or in association.

The results show that the chronic administration of 4-{3-[cis-hexahydrocyclopenta[c]pyrrol-2(1H)-yl]propoxy}benzamide hydrochloride at a dose of 0.3 mg of base/kg per os brings about a moderate anti-hyperalgesic effect because the vocalisation threshold, although increased compared to that of the carrier group, remains lower than the threshold measured before the procedure.

The chronic administration of agomelatine at a dose of 10 mg/kg i.p., for its part, has a moderate and very inconstant anti-hyperalgesic effect, the measured vocalisation threshold measured each day before administration in fact being greatly lowered compared to the threshold measured 45 minutes after adminstration, even clearly approaching the vocalisation threshold of the carrier group.

In contrast, administration of the association of 4-{3-[cis-hexahydrocyclopenta[c]pyrrol-2(1H)-yl]propoxy}benzamide hydrochloride (0.3 mg of base/kg per os) with agomelatine (10 mg/kg i.p.) causes a significantly stronger anti-hyperalgesic effect than that obtained with the compounds administered on their own and which remains constant or even increases over time to allow complete reversal of the hyperalgesia only 4 days after chronic co-administration, the vocalisation threshold in fact becoming equivalent to that measured before the procedure. The increase and the persistance of the anti-hyperalgesic effect observed for the association shows synergy of activity of the two compounds when they are co-administered. Moreover, the biological analyses carried out in this study showed that there was no pharmacokinetic interaction between the two treatments which might explain or play a part in the synergistic effect described hereinbefore.

This powerful and lasting effect of the association is of great importance in the clinical context for dealing with the pain of patients in a constant manner.

### EXAMPLE E

### Effect of 4-{3-[cis-hexahydrocyclopenta[c]pyrrol-2(1H)-yl]propoxy}-benzamide on Anxiety in a Model of Neuropathy Caused by Constriction of the Sciatic Nerve

The effects of 4-{3-[cis-hexahydrocyclopenta[c]pyrrol-2(1H)-yl]propoxy}benzamide were studied on the anxiety associated with the neuropathic pain caused by chronic constriction (CCI) of the sciatic nerve in the rat.

In this study, the rats are placed for 5 minutes in a cylindrical corridor which has two parts closed off by a wall and two opposite parts which are open. The test creates a conflict between the rodent's natural desire to explore and its fear of open spaces which make it vulnerable to predators. Anxiety is measured in this test by the time spent by the rat in exploring the open parts of the cylindrical corridor (Bleickardt et al. Psychopharmacology (Berl), 2009, 202(4):711-8; Alba-Delgado et al. Biol. Psychiatry, 2013, 73(1):54-62).

The results of this study show that, 4 weeks after constriction of the sciatic nerve, the time spent by the rat in the open parts is greatly reduced, compared to those rats which have not undergone constriction of the sciatic nerve. This demonstrates that the neuropathic pain brought about in these rats is associated with anxious behaviour.

### EXAMPLE F

### Effect of 4-{3-[cis-hexahydrocyclopenta[c]pyrrol-2(1H)-yl]propoxy}-benzamide on Depression in a Model of Neuropathy Caused by Constriction of the Sciatic Nerve

The effects of 4-{3-[cis-hexahydrocyclopenta[c]pyrrol-2(1H)-yl]propoxy}benzamide were studied on the depression associated with the neuropathic pain caused by CCI in the rat. In this study, the rat is placed for 5 minutes in a cylinder filled with water, out of which it is unable to climb. After struggling in the water, the animal becomes immobile, reflecting “behavioural despair”, which occurs when the animal realises that it will not be able to escape. Depressive behaviour is measured in this test by the duration of immobility.

The results of this study show that 4 weeks after constriction of the sciatic nerve, the rat's duration of immobility, when it is placed in a cylinder filled with water, is greatly increased compared to rats which have not undergone constriction of the sciatic nerve. This demonstrates that the neuropathic pain brought about in these rats is associated with depressive behaviour.

In conclusion, these test Examples show that 4-{3-[cis-hexahydrocyclopenta[c]pyrrol-2(1H)-yl]propoxy}benzamide, in addition to having notable antalgic properties, has beneficial effects on the anxiety and depression associated with the neuropathic pain caused by constriction of the sciatic nerve in the rat. These results are encouraging in the clinical context where patients suffering from neuropathic pain frequently have psychobehavioural symptoms.

### EXAMPLE G

### Pharmaceutical Composition

Formula for the Preparation of 1000 Tablets Each Containing 5 mg of Active Ingredient (in Terms of the Base Equivalent):

| | |
|---|---|
| 4-{3-[cis-Hexahydrocyclopenta[c]pyrrol-2(1H)-yl]propoxy} | 5.63 g |
| benzamide hydrochloride | |
| Maize starch | 20 g |
| Maltodextrin | 7.5 g |
| Colloidal silica | 0.2 g |
| Sodium starch glycolate | 3 g |
| Magnesium stearate | 1 g |
| Lactose | 65 g |

1-24. (canceled)

25- A method of treating a condition selected from neuropathic pain and cognitive disorders, depression and anxiety associated with that pain in a subject in need thereof, comprising administration of 4-{3-[cis-Hexahydrocyclopenta[c]pyrrol-2(1H)-yl]propoxy}benzamide of formula (I): 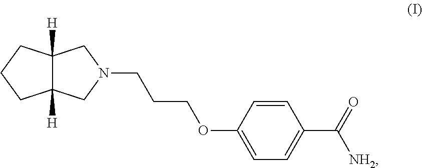 [SMILES: NC(=O)c1ccc(OCCCN2C[C@H]3CCC[C@H]3C2)cc1]
or an addition salt thereof with a pharmaceutically acceptable acid or base.

26- The method according to claim 25, wherein the neuropathic pain is central or peripheral.

27- The method according to claim 26, wherein the peripheral neuropathic pain is selected from metabolic, post-traumatic, post-surgical, toxic or infectious neuropathic pain, pain associated with hereditary neuropathies and with trigeminal neuralgia, and neuropathic pain associated with inflammatory diseases, with certain cancers, and with autoimmune or haematological disorders.

28- The method according to claim 27, wherein the metabolic neuropathic pain is diabetic neuropathic pain.

29- The method according to claim 27, wherein the toxic neuropathic pain is associated with the administration of anticancer medicaments.

30- The method according to claim 26, wherein the central neuropathic pain originates from lesions of vascular, tumour, trauma or inflammatory origin or which are the consequence of neurodegenerative disorders.

31- The method according to claim 25, wherein the 4-{3-[cis-hexahydrocyclopenta[c]pyrrol-2(1H)-yl]propoxy}benzamide of formula (I) is administered as its hydrochloride salt in crystalline form I.

32- The method according to claim 25, wherein the 4-{3-[cis-hexahydrocyclopenta[c]pyrrol-2(1H)-yl]propoxy}benzamide of formula (I), or an addition salt thereof with a pharmaceutically acceptable acid or base, is administered as a pharmaceutical composition comprising the 4-{3-[cis-hexahydrocyclopenta[c]pyrrol-2(1H)-yl]propoxy}benzamide of formula (I), or an addition salt thereof with a pharmaceutically acceptable acid or base, in combination with one or more pharmaceutically acceptable excipients.

33- The method according to claim 32, wherein the pharmaceutical composition comprises 2 mg, 5 mg, 15 mg, 20 mg, 30 mg or 50 mg (expressed in terms of the free base) of 4-{3-[cis-hexahydrocyclopenta[c]pyrrol-2(1H)-yl]propoxy}benzamide.

34- A combination comprising 4-{3-[cis-Hexahydrocyclopenta[c]pyrrol-2(1H)-yl]propoxy}benzamide of formula (I): 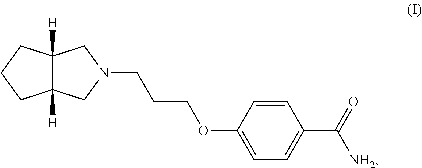 [SMILES: NC(=O)c1ccc(OCCCN2C[C@H]3CCC[C@H]3C2)cc1]
or an addition salt thereof with a pharmaceutically acceptable acid or base and pregabalin.

35- A method of treating a condition selected from neuropathic pain and cognitive disorders, depression and anxiety associated with that pain in a subject in need thereof, comprising administration of the combination according to claim 34.

36- A pharmaceutical composition comprising a combination according to claim 34, in combination with one or more pharmaceutically acceptable excipients.

37- A combination comprising 4-{3-[cis-Hexahydrocyclopenta[c]pyrrol-2(1H)-yl]propoxy}benzamide of formula (I): 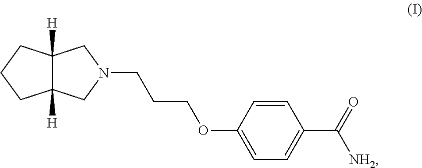 [SMILES: NC(=O)c1ccc(OCCCN2C[C@H]3CCC[C@H]3C2)cc1]
or an addition salt thereof with a pharmaceutically acceptable acid or base and gabapentin.

38- A method of treating a condition selected from neuropathic pain and cognitive disorders, depression and anxiety associated with that pain in a subject in need thereof, comprising administration of the combination according; to claim 37.

39- A pharmaceutical composition comprising a combination according to claim 37, in combination with one or more pharmaceutically acceptable excipients.

40- A combination comprising 4-{3-[cis-Hexahydrocyclopenta[c]pyrrol-2(1H)-yl]propoxy}benzamide of formula (1): 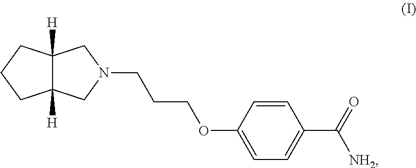 [SMILES: NC(=O)c1ccc(OCCCN2C[C@H]3CCC[C@H]3C2)cc1]
or an addition salt thereof with a pharmaceutically acceptable acid or base and an antidepressant.

41- A method of treating a condition selected from neuropathic pain and cognitive disorders, depression and anxiety associated with that pain in a subject in need thereof, comprising administration of the combination according to claim 40.

42- The combination according to claim 40, wherein the antidepressant is duloxetine, amitriptyline or agomelatine.

43- A pharmaceutical composition comprising a combination according to claim 40, in combination with one or more pharmaceutically acceptable excipients.
